(19) United States
(12) Patent Application Publication
(10) Pub. No.: US 2005/0059140 A1
(43) Pub. Date: Mar. 17, 2005

(54) Methods of surface modification to enhance cell adhesion

(76) Inventors: Andrea Liebmann-Vinson, Willow Springs, NC (US); Bryce N. Chaney, Durham, NC (US)

Correspondence Address: CASTELLANO MALM FERRANO & BUCK PLLC, 2121 K STREET, NW, SUITE 800, WASHINGTON, DC, 20037, US

(21) Appl. No.: 10/660,759

(22) Filed: Sep. 12, 2003

(51) Int. Cl. C12N 11/16

(52) U.S. Cl. 435/289.1; 427/2.14

(57) ABSTRACT

The current invention relates to methods of producing a surface with enhanced cell-adhesive properties comprising treating a pre-formed surface such that at least one intermediate reactive group is exposed on the surface. The exposed intermediate reactive group is then reacted to create a self-assembled monolayer, comprising at least one reactive group. The self-assembled monolayer is created non-mechanically. The reactive group is then coupled to at least one cell-adhesive molecule. The current invention also relates to cell culture devices comprising at least one oxygen-sensing compound and a cell-adhesive molecule.

### BACKGROUND OF THE INVENTION

1. Field of the Invention

The current invention relates to methods of producing a surface with enhanced cell-adhesive properties comprising treating a pre-formed surface such that at least one intermediate reactive group is exposed on the surface. The exposed intermediate reactive group is then reacted to create a self-assembled monolayer, comprising at least one reactive group. The self-assembled monolayer is created non-mechanically. The reactive group is then coupled to at least one cell-adhesive molecule to form the surface with enhanced cell-adhesive properties. The current invention also relates to cell culture devices comprising at least one oxygen-sensing compound and a cell-adhesive molecule.

2. Background of the Invention

Conventional methods for monitoring cell growth, such as measuring cellular DNA with fluorescent dye, measuring cell metabolism or directly counting cells, is invasive, disruptive and may result in non-reproducible values. These end point assays are labor-intensive, and the sample requirements are expensive because different samples are needed at each time point. Thus, end point assays are not useful for monitoring cell growth over time in a high throughput manner.

Another approach to cell culture progress involves the use of oxygen biosensors (OBSs). These devices provide an effective way of monitoring cell growth in cell culture. The oxygen biosensor directly measures cell metabolism, which gives an indirect measure of cell growth. Solid-state, fluorescence-based oxygen sensors are highly sensitive, selective and affordable. Oxygen biosensors can provide a non-invasive real time measurement of cell growth in cell culture.

Typically, oxygen biosensors are based on fluorescent dye crystals that exhibit strong luminescence upon irradiation. The luminescent properties of the fluorescent dye crystals may be efficiently quenched by oxygen, which results in a change in the luminescence signal directly related to the oxygen partial pressure in the environment. Organic ruthenium (II) complexes are popular oxygen sensor dyes owing to their high-quantum yield luminescence, high selectivity, good photostability, and relatively long lifetime.

Cell culture plates with embedded oxygen biosensors (OBS plates) are available from Becton Dickinson and Company (BD); however, the surfaces of these devices often do not support cell adhesion, even in the presence of serum, such that the OBS plates are generally useful only with non-adherent cell cultures. Passively coating the OBS plates with cell adhesion molecules can not cure this problem of non-adhesion because the culture media will often hydrate the cell adhesion molecules, thus freeing them into the cell culture media and removing them from the surface. Additionally, a three-dimensional cell culture scaffold may not always be desired or needed. Thus, there remains a need for a two-dimensional, cell-adhesive cell culture surface capable of monitoring cellular metabolism through the use of oxygen biosensors.

### SUMMARY OF THE INVENTION

The current invention relates to methods of producing a surface with enhanced cell-adhesive properties comprising treating a pre-formed surface such that at least one intermediate reactive group is exposed on the surface. The exposed intermediate reactive group is then reacted to create a self-assembled monolayer, comprising at least one reactive group. The self-assembled monolayer is created non-mechanically. The reactive group is then coupled to at least one cell-adhesive molecule to form the surface with enhanced cell-adhesive properties. The current invention also relates to cell culture devices comprising at least one oxygen-sensing compound and a cell-adhesive molecule.

### BRIEF DESCRIPTION OF THE DRAWINGS

FIG. 1A is a graph showing the percentages of elements composing the surface of derivatized silicon wafer and PDMS samples; FIG. 1B is a graph showing the elemental ratios of the elements composing the surface of derivatized silicon wafer and PDMS samples.

FIGS. 2A-2D are microscopy images of cells attached to the bottom of tissue culture dishes to the surface of PDMS derivatized with coupled fibronectin, as described in Example 4.

FIGS. 3A-D are photomicrographs of MC3T3 cells cultured on PDMS plus ECM, 10% FCS, hematoxylin on various surfaces, as indicated, as described in Example 4.

FIG. 4 depicts a graph of fluorescence in normalized fluorescence units (NRFU) from HepG2 cultures on a modified OBS plate (round symbols) and an unmodified OBS plate (square symbols). Each data point represents an average reading from 60 wells.

### DETAILED DESCRIPTION OF THE INVENTION

The current invention relates to methods of producing a surface with enhanced cell-adhesive properties comprising treating a pre-formed surface such that at least one intermediate reactive group is exposed on the surface. The exposed intermediate reactive group is then reacted to create a self-assembled monolayer, comprising at least one reactive group. The self-assembled monolayer is created non-mechanically. The reactive group is then coupled to at least one cell-adhesive molecule to form the surface with enhanced cell-adhesive properties.

The methods of the invention result in a surface suitable for cell culture with enhanced cell-adhesive properties. As used herein, “enhanced cell-adhesive properties” is a relative term that requires comparing the cell-adhesive properties of the pre-formed surface with the cell-adhesive properties of the surface after performing the methods of the invention described herein. A “cell-adhesive property” is a property of the surface and is used to indicate the ability of live cells, or a live cell, to adhere or attach to the surface and be able to, at a minimum, withstand gentle sheer forces, e.g., gentle shaking, or removal and/or addition of culture media, without detaching. Of course, a surface's cell-adhesive properties may be stronger such that the attached live cells or cell may be able withstand virtually any amount of sheer force without detaching. As used herein, a live cell is a cell that is metabolically active. A live cell need not actively grow or actively divide, provided the cell is metabolically active.

Thus, the methods of the current invention increase the ability of live cells to attach or remain attached to the pre-formed surface after treatment. The increase in the ability of the cells to adhere can be examined in a number of ways including, but not limited to, examining the number of adherent cells with increasing sheer forces and performing competition assays. The pre-formed surface may possess cell-adhesive properties prior to the application of the methods described herein, or the surface may not possess any cell-adhesive properties prior to the methods described herein.

The pre-formed surface should be suitable for culturing cells at least after performing the methods of the current invention described herein; however, the pre-formed surface may also, but need not be, suitable for culturing cells prior to applying the methods of the current invention. “Pre-formed” is used to indicate a surface that is to be subjected to the methods of the invention described herein. Examples of material comprising the pre-formed surface include, but are not limited to, glass, nylon, plastic, silicon, polystyrene, polypropylene, polycarbonate, polymethacrylate, polyvinylchloride and latex. The pre-formed surface can be horizontal, vertical or sloped. The pre-formed surface can be rounded or flat, smooth or ridged. Examples of preformed surface include, but are not limited to, microtiter wells, culture flasks, petri dishes, and glass or plastic slides.

In one embodiment, the pre-formed surfaces comprise a polymer. Examples of polymers that comprise the pre-formed surface include, but are not limited to, polyorganosiloxanes. Polyorganosiloxanes are linear and cyclic compounds such as, but not limited to, dimethylvinylsilyl-terminated dimethylpolysiloxanes, trimethylsilyl-terminated (methylvinyl)(dimethyl)polysiloxane copolymers, dimethylvinylsilyl-terminated (methylvinyl)(dimethyl)polysiloxane copolymers and cyclic methylvinylpolysiloxanes.

In another embodiment of the current invention, the pre-formed surface further comprises an oxygen-sensing particles or compounds, in addition to a polymer. In this embodiment, the oxygen-sensing compound may or may not be luminescent. Examples of luminescent oxygen-sensing particles include, but are not limited to, any salt of tris-4,7-diphenyl-1,10-phenanthroline ruthenium (II), any salt of tris-2,2′-bipyridyl-ruthenium (II), any salt of tris-1,7-diphenyl-1,10 phenanthroline ruthenium (II), and 9,10-diphenyl anthracene. Luminescent particles can also include platinum (II) octaethyl porphyrin complexes, palladium (II) octaethyl porphyrin complexes, palladium-meso-tetra(4-carboxyphenyl)porphine, palladium-meso-tetra(4-carboxyphenyl)porphyrin dendrimer and palladium-meso-tetra(4-carboxyphenyl)tetrabenzoporphyrin dendrimer.

Examples of salts of tris-4,7-diphenyl-1,10-phenanthroline ruthenium (II) include, but are not limited to, tris-4,7-diphenyl-1,10-phenanthroline ruthenium (II) dichloride pentahydrate, tris-4,7-diphenyl-1,10-phenanthroline ruthenium (III) trichloride, tris-4,7-diphenyl-1,10-phenanthroline ruthenium (II) diperchlorate and tris-4,7-diphenyl-1,10-phenanthroline ruthenium hexafluorophosphate. An example of a salt of tris-2,2′-bipyridyl-ruthenium (II) includes, but is not limited to, tris-2,2′-bipyridyl-ruthenium (II) chloride hexahydrate. An example of a salt of tris-1,7-diphenyl-1,10 phenanthroline ruthenium (II) includes, but is not limited to, tris-1,7-diphenyl-1,10 phenanthroline ruthenium (II) dichloride.

The methods of the current invention require treating the pre-formed surface to expose at least one intermediate reactive group on the pre-formed surface. As used herein, “exposed” is used to indicate the creation or formation of intermediate reactive groups at or near the top of the pre-formed surface. “Exposed” is also used to indicate the uncovering or surfacing of protected or covered intermediate reactive groups at or near the top of the pre-formed surface.

The term “intermediate reactive group” is used to indicate a chemical group that can be reactive or made reactive through chemical or physical modification. Examples of intermediate reactive groups include, but are not limited to, a carboxyl group, an amine and a hydroxyl group.

The methods of the current invention comprise exposure of the intermediate reactive groups on the pre-formed surface. The exposure of the intermediate reactive groups can include one or multiple reactions. The exposure reaction(s) should be tailored by the types of reactive and/or intermediate reactive group(s) sought, as well as the material comprising the pre-formed surface. For example, hydroxyl groups on a pre-formed surface comprising polydimethyl siloxane (PDMS) can be exposed by treating the plate with UV radiation or a combination of UV radiation and ozone generation known as UV/Ozone (UVO) treatment. UVO treatment can be carried out in a commercially available UVO chamber (Jelight Company, Inc.). Briefly, during UVO treatment, the molecules of surface material are dissociated (or excited) by short wavelength (higher frequency) ultraviolet radiation; while atomic oxygen is also generated simultaneously from molecular oxygen and ozone. The polymer radicals created at the surface can either recombine to form networks with surface properties similar to UV-treated PDMS, or the polymer radicals can recombine with the atomic oxygen to form hydroxyl groups, thus rendering the surface hydrophilic. (See Efimenko, K., et al., J. Colloid Interface Sci., 254(2): 306-315 (2002), which is hereby incorporated by reference). Other treatment reactions include physical or chemical treatments, or combinations thereof. For descriptions of treatment reactions or procedures, please see Chaudhury, M. K., et al. Langmuir, 7:1013-1025 (1991); Lee, S. D., et al., Journal of polymer Science: Part A: Polymer Chemistry, 34:141-148 (1996); Hsiue G. H., et al., J. Biomed. Mater. Res., 42: 134-147, (1998); Silver, J. H., et al., Journal of Biomedical Materials Research, 29: 535-548 (1995); Silver et al., Biomaterials, 20: 1533-1543 (1999) and Lateef S. S, et al., Biomaterials, 23: 3159-3168 (2002), which are hereby incorporated by reference. Examples of additional forms of treatment include, but are not limited to, using oxygen plasma, water plasma, and plasma graft-polymerization.

Treating the preformed surface with glutaraldehyde can also expose and activate reactive groups. For example, treating with glutaraldehyde at temperatures between 0° C. and 80° C. will couple the glutaraldehyde with any exposed nucleophilic groups, such as amines, thiols and hydroxyls.

Treating the pre-formed surface with cyanuric chloride can also expose and activate reactive groups. For example, treating with a nonaqueous solution of about 5% cyanuric chloride for about one hour, at temperatures between 0° C. and 80° C., will activate the cyanuric chloride with exposed nucleophilic groups, such as amines, thiols and hydroxyls. Any nucleophilic groups on the cell-adhesive molecules, such as thiols, amines, and hydroxyls, can then be coupled to the surface-coupled cyanuric chloride. Similarly, treating the surface with other sulfonyl chlorides can also expose and activate reactive groups.

Treating the surface with cyanogen bromide can also expose and activate reactive groups. For example, treating with an aqueous solution (pH of about 10-11) of cyanogen bromide at temperatures about 20° C. or below will covalently couple and activate surface hydroxyl groups. The exposed and activated reactive groups can then be coupled with the cell-adhesive molecule via any amines on the peptide.

Treating the surface with succinic anhydride can also expose and activate active carbonyl-bearing esters. For example, succinic anhydride can react with surface hydroxyls and amines to produce esters and amides, respectively. The exposed and activated reactive groups can then be coupled to the cell-adhesive molecule via amine or hydroxyl groups.

Another example of exposing the intermediate reactive groups includes such reactions as adding the intermediate reactive group (or potential intermediate reactive group) to the surface prior to activation. For example, preactivation may comprise “preactivating” a polymer by adding hydroxyls with benzoin dimethyl sulfoxide. Hydroxyl groups can be added to poly(tetrafluoroethylene) (PTFE) as described by Costello and McCarthy, Macromolecules 20:2819-2828 (1987), which is hereby incorporated by reference. Briefly, benzoin is added to a solution of potassium tert-butoxide in dimethyl sulfoxide and placed in contact with the PTFE surface. The reaction is allowed to proceed at 50° C. for about 1 hour. The material is removed and rinsed with tetrahydrofuran (THF). This preactivated surface is then treated with 1M borane in THF at room temperature for about 12 hours. The surface is then treated with 1M NaOH containing 10% hydrogen peroxide at about 0° C. for about 3 hours, after which the surface is washed sequentially with dilute NaOH, water, dilute HCl, water, THF, and heptane. This preactivation reaction produces a surface that is rich in hydroxyls and that can subsequently be activated by several methods including, but not limited to any of the chemistries described herein.

The methods of the current invention comprise reacting the intermediate reactive groups to produce a self-assembled monolayer (SAM). The current invention requires that the self-assembled monolayer be created non-mechanically. As used herein, the creation of a self-assembled monolayer through non-mechanical methods indicates that the polymer matrix that comprises the pre-formed surface is not stretched or otherwise physically altered at any time during the methods of the current invention. In other words, while the polymer comprising the pre-formed surface may be flexible, and thus capable of mechanical, i.e., non-chemical manipulation or alternation, the methods of the current invention rely on the polymer not being mechanically altered during the treatment, reacting and coupling steps of the current invention.

The treatment reaction to expose the intermediate reactive groups and the subsequent reaction to produce the SAM may be performed in the same reaction or in sequential reactions. The reactions and agents used therein for creating the SAM should be specific for the intermediate reactive groups and/or reactive groups. In one embodiment, the SAMs used in the present invention are alkylsilane derivatives, which are represented by the formulae RSiX₃, R₂SiX₂, or R₃SiX, wherein X is chloride or alkoxy and R is a carbon chain containing the functional groups. The exposed functional groups of the SAM may be amines, thiols, pyridyl, carboxyl, vinyl, sulfydryl, or aldehyde groups. In one specific embodiment, the SAM is a chlorosilane-based oligomer or polymer, more preferably, a trichlorosilane-based oligomer or polymer. For example, trichlorosilanes react with hydroxyl groups on an oxidized silicone surface to create covalently attached self-assembled monolayers. (See Silver et al., Biomaterials, 20: 1533-1543 (1999), which is hereby incorporated by reference). Self-assembled monolayers of silanes, with a variety of different functional groups on silicone rubber, can be prepared by methods known to those skilled in the art, e.g., Ulman, A., An Introduction to Ultrathin Organic Films: From Langmuir-Blodgett to Self-Assembly, Academic Press, May 1991, which is hereby incorporated by reference. Examples of terminal functional groups include, but are not limited to, —CH3, —CF3, —COOH, —OH, —NH3 and —CHO.

In one embodiment, the non-mechanical SAM does not comprise a reactive group immediately after deposition onto the preformed surface. In another embodiment, the non-mechanical SAM comprises a reactive group immediately after deposition onto the pre-formed surface. Thus the methods may further comprise an additional conversion reaction to convert a non-reactive terminal group on the non-mechanical SAM to a reactive group on the non-mechanical SAM. For example, a PDMS surface may be reacted with vinyl-terminated silanes, which do not comprise reactive groups. The terminal vinyl groups can then be oxidized to form reactive, functional carboxyl (—COOH) groups.

Continuing with the example, the carboxyl group is then activated, using a carbodiimide, to couple the cell-adhesive molecule to the surface. In one specific embodiment, reacting the intermediate group comprises ethyldimethylaminopropyl-carbodiimide (EDC). Other carbodiimides that can be used include, but are not limited to, 1-cyclohexyl-3-(2-morpholinoethyl)carbodiimide and dicyclohexyl carbodiimide. For other examples of carbodiimides, see Hermanson, G. T., Bioconjugate Techniques, Academic Press, San Diego, (1996), which is hereby incorporated by reference. The concentration of agent used in activating the reactive group can be from about 0.01 mg/ml to about 50 mg/ml. In one embodiment, the concentration of activation agent in solution is from about 0.1 mg/ml to about 10 mg/ml. In another embodiment, the concentration of the activation agent in solution is from about 1 mg/ml to about 5 mg/ml.

In another embodiment, reacting the intermediate reactive groups comprises an agent that will stabilize or protect the exposed reactive groups. Examples of agents that can stabilize or protect the reactive group include, but are not limited to, N-hydroxysulfosuccinimide (sulfo-NHS), hydroxysulfosuccinimide and hydroxybenzotriazolohydrate, which are described in U.S. application Ser. No. 10/259,797, filed Sep. 30, 2002, which is hereby incorporated by reference. The concentration of the stabilizing or protective agent in solution should be at least about 0.01 mg/ml. In one embodiment, the concentration of stabilizing or protective agent in solution is from about 0.1 mg/ml to about 10 mg/ml. In another embodiment, the concentration of the stabilizing or protective agent in solution is from about 1 mg/ml to about 5 mg/ml.

Next, the methods of the current invention comprise coupling at least one cell-adhesive molecule to the activated reactive groups. The coupling to the reactive groups can be any form of bond that attaches the cell-adhesive molecule to the activated, exposed reactive groups, such as, for example, an ionic bond, a hydrogen bond, a metallic bond, a polar bond, and a covalent bond including, but not limited to, peptide bonds.

In one embodiment, the intermediate reactive groups and the reactive groups are different chemical entities. In another embodiment, the intermediate reactive groups and the reactive groups are the same chemical entities.

In one embodiment of the invention, the reactive groups are activated prior to or simultaneously with the coupling of the cell-adhesive molecules. In another embodiment, activation of the reactive groups is not required for the coupling reaction. If the reactive group is an aldehyde group, no activation is necessary. Generally, a reaction between an aldehyde and amine will occur spontaneously.

Because the coupling reaction involves reactive groups, the coupling reaction may involve placing a buffered solution comprising the cell-adhesive molecule onto the surface. Alternatively, the coupling reaction may comprise an additional agent, such as, but not limited to, a reducing and/or oxidizing agent, an acid, a base, a buffer, a catalyst, an organic solvent and an inorganic solvent. Of course, oxidizing agents are atoms, ions, and molecules that have an unusually large affinity for electrons. Examples of oxidizing agents include, but are not limited to, elemental fluorine, F₂, and O₂, O₃, and Cl₂, which are the elemental forms of the second and third most electronegative elements, respectively. Additional oxidizing agents include compounds with unusually large oxidation states, such as the permanganate (MnO₄⁻), chromate (CrO₄²⁻), and dichromate (Cr₂O₇²⁻) ions, as well as nitric acid (HNO₃), perchloric acid (HClO₄), and sulfuric acid (H₂SO₄). These listed compounds are strong oxidizing agents because elements become more electronegative as the oxidation states of their atoms increase.

Of course, reducing agents are atoms, ions and molecules that have a propensity to lose negative charge (electrons), usually in the form of a loss of oxygen and/or a gain of hydrogen. Examples of reducing agents include, but are not limited to, the active metals, such as sodium, magnesium, aluminum, and zinc, which have relatively small ionization energies and low electro-negativities. Additional reducing agents include the metal hydrides, such as NaH, CaH₂, and LiAlH₄. Reducing agents also include reagents such as cyanoborohydrate that can stabilize the reaction product of an aldehyde with an amine, so-called reductive amination.

In one embodiment of the current invention, the coupling reaction comprises a solution of cell-adhesive molecule only. In another embodiment, the coupling reaction comprises a solution of cell-adhesive molecule and a separate solution of a reducing agent. In another embodiment, one solution contains both the cell-adhesive molecule and the reducing agent. Examples of a reducing agent include, but are not limited to, cyanoborohydrate.

As used herein, a cell-adhesive molecule is a molecule that increases adhesiveness of cells either to other cells, e.g., cell-cell adhesion molecules (CAMs), or to substrates or surfaces, e.g., cell-substrate adhesion molecules. Cell-adhesive molecules can be any class of chemical, including, but not limited to, a protein, a protein fragment, a polypeptide, an oligopeptide, an amino acid, a proteoglycan, a glycoprotein, a lipoprotein, a carbohydrate, a disaccharide, a polysaccharide, a nucleic acid, an oligonucleotide, a polynucleotide, a small organic molecule, a small inorganic molecule, a synthetic polymer and a natural polymer. Typical examples of cell-adhesive molecules include, but are not limited to, the major classes of traditional cell adhesion molecules, such as integrins, selecting, cadherins and the immunoglobulin members, as well as growth factors, extracellular matrix molecules, receptors and antibodies, or functional (i.e., binding) fragments thereof.

The major classes of cell adhesion molecules include the integrins, selecting, cadherins and members of the immunoglobulin (Ig) superfamily. Each of the classes of cell adhesion molecules include several, well-known members that are widely recognized in the art. Additionally, new cell-adhesive molecules may be identified by one of several ways, including but not limited to, interactions with antibodies known to disrupt cell adhesion, immunoprecipitation, cloning of putative CAMs or SAMs from expression libraries, binding or competition assays, conjugation of the putative cell-adhesive molecule to microspheres, attachment assays, centrifugal-force assays and transfection experiments. Examples of known cell-adhesive molecules include, but are not limited to, E-cadherin, P-cadherin, N-cadherin, B-cadherin R-cadherin, EP-cadherin, OB-cadherin, M-cadherin, cadherin-5, cadherin-12, protocadherin 43, desmocollin 1, desmoglein 1, α₁β₁integrin, α₂β₁integrin, α₃β₁integrin, α₄β₁integrin, α₅β₁integrin, α_{V}β₁integrin, α_{L}β₂integrin, α_{M}β₂integrin, α_{V}β₃integrin, P-selectin, E-selectin, intercellular adhesion molecule 1 (ICAM 1), ICAM 2, neural cell adhesion molecule (NCAM), platelet-endothelial cell adhesion molecule (PECAM), vascular adhesion molecule (VCAM), carcinoembryonic antigen and L1.

Examples of antibodies, or functional fragments thereof, that are cell-adhesive molecules include, but are not limited to, CD34, CD133 and CD38. Examples of suitable growth factors include, but are not limited to, basic fibroblast growth factor (bFGF) and vascular endothelial growth factor (VEGF).

Extracellular matrix (ECM) molecules include, but are not limited to, the two major classes of ECM molecules: glycosaminoglycans and fibrous proteins. Glycosaminoglycans are polysaccharide chains that are usually covalently linked to a protein or proteoglycan. The fibrous proteins include, but are not limited to, collagen types, I, II, III, IV, V and VI, elastin, laminin and fibronectin.

The amount of cell-adhesive molecule used in the coupling reaction will vary, depending on several factors, including, but not limited to, the concentration or density of exposed, activated reactive groups on the surface, the desired increase in adhesiveness of the resulting surface, the chemical and physical properties of the cell-adhesive molecule, and the conditions (e.g., temperature, pressure, time and pH) under which the methods of the current invention take place.

The methods of the current invention encompass using one or more cell-adhesive molecules. For example, more than one type of cell-adhesive molecule, e.g., collagen I and collagen IV, may be coupled to the reactive groups, or a single type of cell-adhesive molecule, e.g., collagen VI, can be coupled to the reactive groups.

The examples presented herein are meant for illustrative purposes only and are not intended to limit the scope of the subject matter described herein.

### EXAMPLES

### Example 1

### Preparation of a Modified Cell Culture Surface

Treatment: An oxygen biosensor plate (BD) which comprises a luminescent dye (4,7,-diphenyl-1,10-phenanthroline ruthenium II chloride) embedded in polydimethyl siloxane (PDMS), was UVO treated, followed by chemical treatment to create a carboxyl-terminated self-assembled monolayer, according to the methods of Efimenko, K., et al., J Colloid Interface Sci., 254(2): 306-315 (2002).

Activation: Carboxyl groups exposed on the PDMS surface were activated using ethyldimethylaminopropyl-carbodiimide (EDC) in the presence of N-hydroxysulfosuccinimide (sulfo-NHS) to stabilize the hydrolytically instable active ester (o-acylisourea) intermediate. A 2 mg/ml solution of both EDC and sulfo-NHS in 2-[N-Morpholino]ethane sulfonic acid (MES) buffer was used for this activation step. Surfaces were activated for about 5 minutes before continuing with the coupling step.

Coupling: Collagen type VI was covalently coupled to the activated carboxyl groups on the PDMS surface by adding a given volume of a 100 ug/ml solution containing Collagen type VI in acetic acid (10 mM) buffer to the activated PDMS surfaces (it was not necessary to remove the EDC/NHS solution). The stabilized activated intermediate groups introduced by the EDC/NHS solution facilitate the formation of amide bonds between the amine groups on the collagen and the carboxyl groups on the silicone surface. The final protein concentration in the coupling solution was 50 ug/ml. Coupling was allowed to proceed overnight. The coupled surfaces were removed from the coupling solution the following day, and washed thoroughly with copious amounts of deionized water and dried before cell culture.

### Example 2

### Modifying Silicon with Laminin

One silicon wafer sample (Wacker Siltronix Corporation, Portland, Oreg.) and 2 different PDMS samples bearing chlorosilane based self-assembled monolayers with —COOH terminal groups were provided as follows:

Sample 1: Silicon wafer; Sample 2: duplicate of Sample 1; Sample 3: PDMS sample 1; Sample 4: duplicate of Sample 3; Sample 5: PDMS sample 2; Sample 6: duplicate of Sample 5.

The —COOH groups were activated by adding a solution containing 4 mg/ml 1-ethyl-3-(3-dimethylaminopropyl)carbodiimide hydrochloride (EDC) and N-hydroxysulfosuccinimide (sulfo-NHS) and letting the sample soak in the EDC/sulfo-NHS solution for about 5 minutes. Then a 200 μg/ml mouse laminin solution was added, and the samples were left soaking in the lamin/EDC/sulfo-NHS solution overnight for the coupling reaction to take place. Samples were removed from the laminin/EDC/sulfo-NHS solution the next morning, rinsed first with a salt/AcOH/water (40 ml 5M NaCl/2 ml acetic acid/158 ml deionized water) mixture to remove any non-specifically adsorbed laminin followed by thorough washing with deionized water (DIH₂0).

Samples were left to air dry and were then taken for ESCA (electron spectroscopy for chemical analysis, also called XPS) analysis. The presence or absence of nitrogen in the ESCA survey scan can be used as indicator for the presence or absence of laminin coupled to the silicon surface. Theoretically, no nitrogen is present prior to the immobilization of laminin, but is introduced on the surface only through immobilized laminin. The elemental surface compositions are summarized in Table 1.

TABLE 1
Elemental surface composition of derivatized silicon
wafer and PDMS surfaces

| | C | O | N | Si | Others |
|---|---|---|---|---|---|
| Sample 1 | 63.58 | 21.37 | 13.25 | 1.79 | |
| Sample 2 | 64.00 | 20.24 | 12.14 | 3.63 | |
| Sample 3 | 57.28 | 23.74 | 8.99 | 9.47 | Br 0.52 |
| Sample 4 | 56.03 | 24.81 | 7.73 | 10.61 | Br 0.82 |
| Sample 5 | 60.39 | 22.62 | 9.87 | 7.12 | |
| Sample 6 | 60.27 | 22.41 | 8.51 | 8.81 | |

As indicated in Table 1, nitrogen was observed on every surface derivatized with laminin indicating that laminin was in fact coupled to the silicon wafer and PDMS surface.

### Example 3

### Modifying Silicon with Fibronectin

The following flexible polymer matrix had surfaces prepared as follows:

Sample A-1 (silicon wafer)—as received from the manufacturer (i.e., untreated); Sample B-1 (PDMS 1)—untreated; Sample C-1 (PDMS 2)—untreated

Sample A-2 (silicon wafer)—UVO-treated; Sample B-2 (PDMS 1)—UVO-treated; Sample C-2 (PDMS 2)—UVO treated;

Sample A-3 (silicon wafer)—silicon with SAM; Sample B-3 (PDMS 1)—silicon with SAM; Sample C-3 (PDMS 2)—silicon with SAM.

Sample A-4 (silicon wafer)—oxidized terminal (vinyl) group using potassium permanganate (KMnO₄); Sample B-4 (PDMS 1-28 kDa)—oxidized; Sample C-4 (PDMS 2-17.2 kDa)—oxidized;

The oxidized samples were rinsed with ethanol and water, then activated with an aqueous solution containing 4 mg/ml containing EDC/sulfo-NHS for approximately 5 minutes after which an equal amount of 100 μg/ml containing human fibronectin (Fn) solution was added. The samples were left on the bench overnight for the coupling reaction to take place.

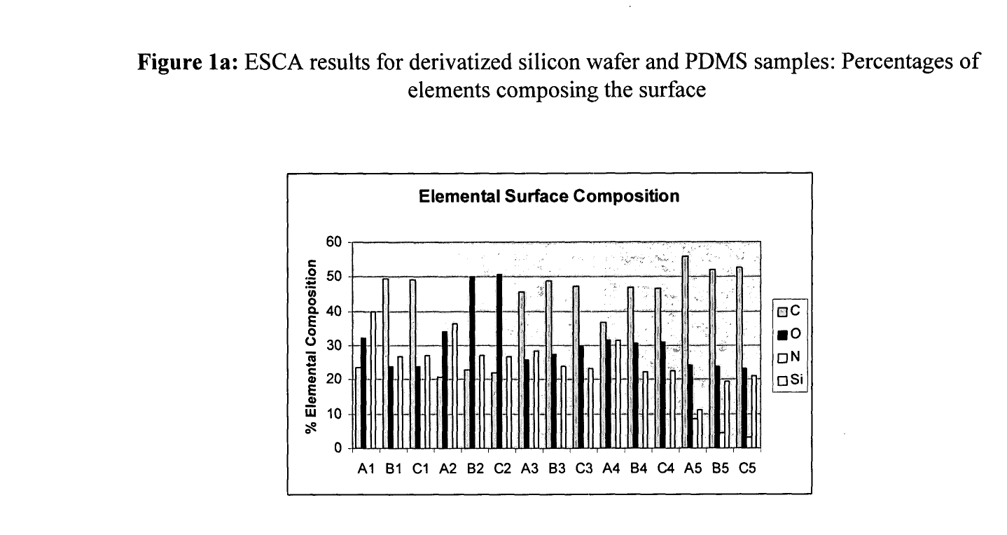

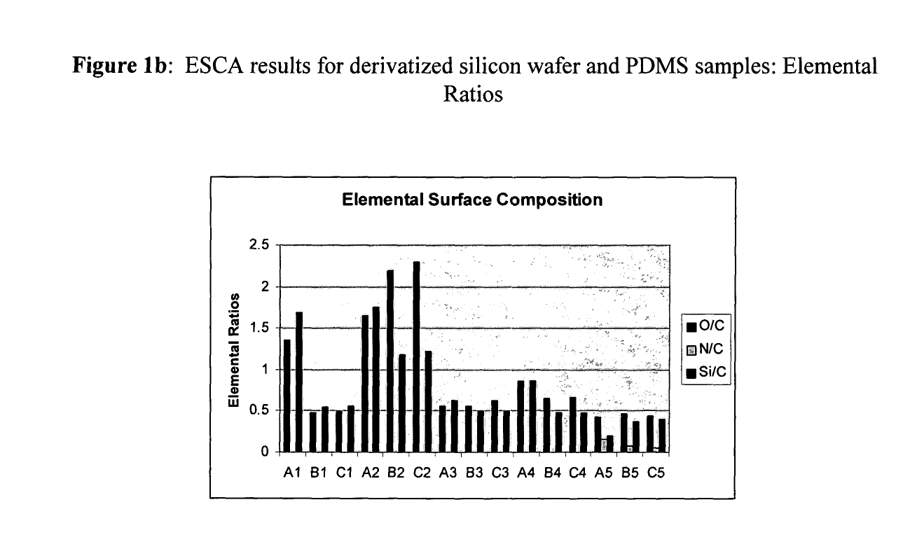

The following morning, the samples were removed from the solutions, rinsed in salt/AcOH/water (40 ml 5M NaCl/2 ml acetic acid/158 ml deionized water) mixture solution to remove any non-specifically bound fibronectin, followed by thorough rinsing with DIH₂O. The samples were then air dried. Surface chemical compositions of samples at each surface derivatization step were determined by ESCA. The results of this analysis were summarized in Table 2 and shown in FIG. 1a and 1b.

TABLE 2
Elemental surface composition of derivatized silicon
wafer and PDMS surfaces

| | C | O | N | Si | Others |
|---|---|---|---|---|---|
| Sample A-1 | 23.56 | 32.09 | | 39.94 | |
| Sample B-1 | 49.38 | 23.91 | | 26.71 | |
| Sample C-1 | 48.99 | 23.93 | | 27.07 | |
| Sample A-2 | 20.7 | 34.07 | | 36.39 | |
| Sample B-3 | 22.84 | 50.15 | | 27.03 | |
| Sample C-2 | 22.15 | 50.88 | | 26.96 | |
| Sample A-3 | 45.74 | 25.80 | | 28.46 | |
| Sample B-3 | 48.76 | 27.44 | | 23.79 | |
| Sample C-3 | 47.25 | 29.54 | | 23.20 | |
| Sample A-4 | 36.68 | 31.60 | | 31.72 | |
| Sample B-4 | 46.91 | 30.65 | | 22.44 | |
| Sample C-4 | 46.58 | 30.91 | | 22.51 | |
| Sample A + Fn | 55.89 | 24.12 | 8.74 | 11.26 | |
| Sample B + Fn | 52.05 | 23.98 | 4.43 | 19.55 | |
| Sample C + Fn | 52.56 | 23.32 | 3.04 | 21.08 | |

As indicated in Table 2, nitrogen was only introduced in the Fn immobilization step and is thus a good indicator that Fn was coupled to the silicon wafer and PDMS surfaces. In addition, it could be noted that the Si signal from the underlying silicon wafer or PDMS substrate is increasingly attenuated first by the addition of the self-assembled monolayer coating and even more when Fn is coupled to the surfaces. This is consistent with the addition of material on top of the silicon wafer or PDMS surface considering that when analyzing surfaces using ESCA at a 35° electron take-off angle, the sampling depth is constant at about 65.

### Example 4

### Cell Culture on the Silicon-Fibronectin Surface

Cell culture was performed to test the biological activity of the immobilized Fn. MC3T3-El osteoblast cells (comparable cells are available from the ATCC; other cells having attachment properties are also suitable) were grown using standard cell culture techniques. MC3T3-El is a well-characterized and rapidly growing osteoblast cell line that was chosen because it attached aggressively to most commonly used tissue culture surfaces.

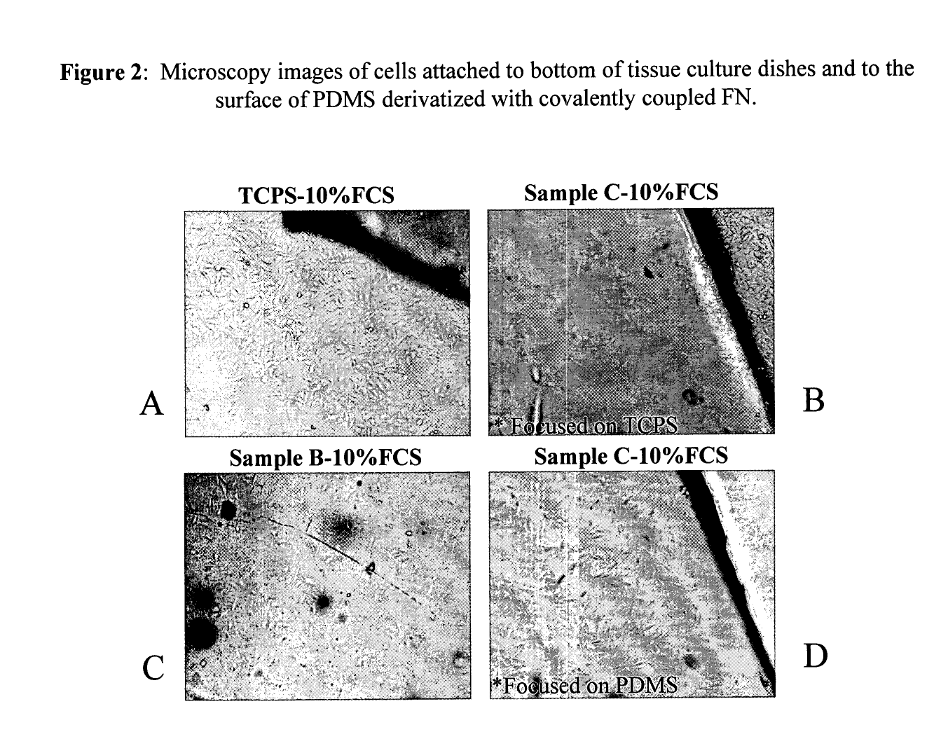

Cells were removed from cell culture flasks using trypsin-EDTA. Cells were counted, spun down and re-suspended in media containing 10% fetal calf serum. Pieces of the derivatized surfaces were stuck to the bottom of a 60 mm tissue culture dish using a small amount of vacuum grease. Cells were seeded onto the derivatized surfaces at a seeding density of about 10⁶ cells per 60 mm culture dish. Cells were allowed to attach and grow for about 4 hours before the culture was studied by light microscopy. Cell attachment was observed on the bottom of the 60 mm culture dish as well as on top of the derivatized PDMS surfaces, as is shown in FIG. 2.

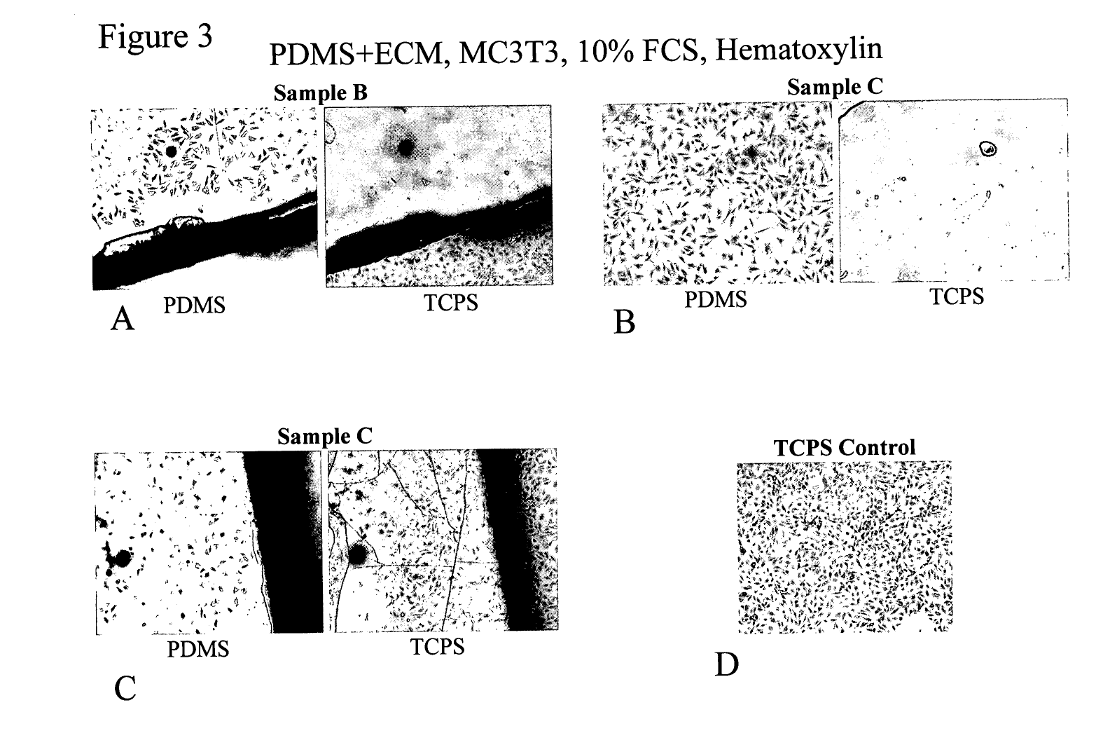

Because cell attachment could not be visualized using light microscopy on top of the silicon wafers, the cells were fixed in formalin and stained purple using hematoxylin stain. Attached cells were clearly visible as a purple coating on the silicone wafers. Also, attached cells were clearly visible on the derivatized PDMS samples, as shown in FIG. 3.

### Example 5

### Cell Culture on a Modified and Unmodified Cell Culture Surfaces

Cell Culture: HepG2 cells (human hepatoma) available from the ATCC were seeded onto their inner 60 wells of a modified and an unmodified OBS plate. Initial cell density was about 3,000 cells/well. The growth media was DMEM, sodium pyruvate, nonessential amino acids+10% fetal calf serum, with pen/strep/fungizone. Plates were read on a BMG Polarstar fluorometer set at 37° C. Ruthenium fluorescence was read at an excitation of 465 nm and emission at 590 nm at the indicated time points. Data are the average of all 60 inner well reads, and error bars represent standard deviation. Normalized fluorescence was derived by dividing the initial fluorometer values by the subsequent values, giving the overall change in fluorescence in the well over time that is independent of the initial fluorescence of the ruthenium dye.

Commonly, HepG2 cells seeded on a round-bottom 96-well OBS plate form a single large clump in the center (bottom) of the well, indicating a lack of adhesion to the surface of the bottom of the plate. In comparison, although the surface in a round-bottom plate is not flat and thus does not present an ideal surface for adherent cell culture, a number of small HepG2 clumps nonetheless attached to the modified surface. The fact that cells adhered in small clumps in these wells demonstrates adhesiveness of these HepG2 cells to the modified surfaces. The cells on the untreated surface are present in a single cell plug at the bottom of the OBS plate:

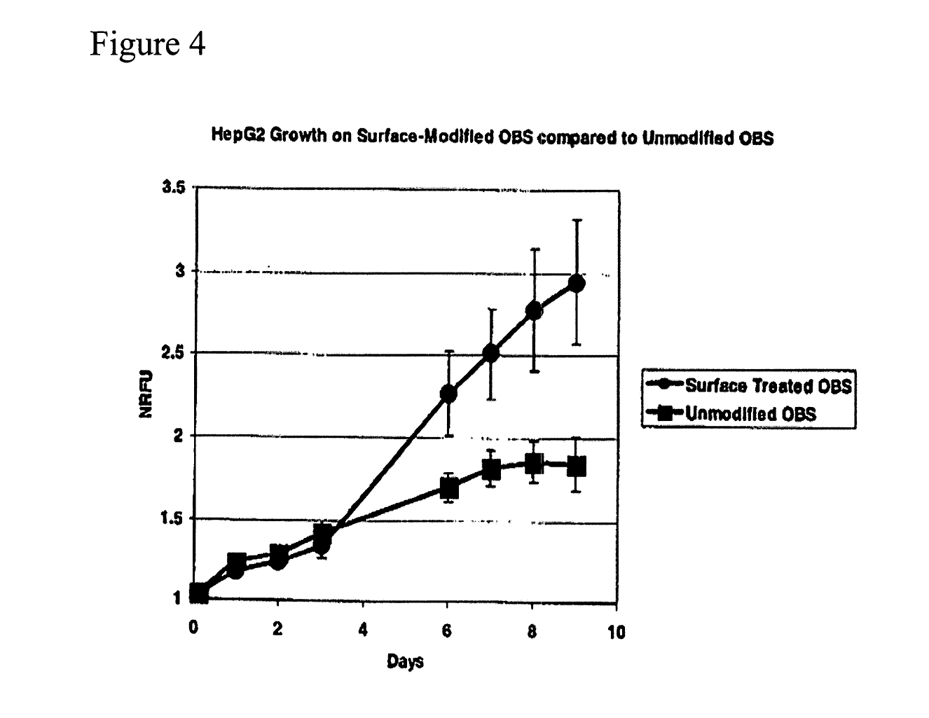

The fluorescent signal from the unmodified surface of the OBS plate on which the HepG2 cells were seeded shows a steady signal but does not increase over time, indicating that the cells were contact inhibited, i.e., the cells were consuming a basal level of oxygen but not proliferating. This contact inhibition behavior of the cells on the OBS plate is consistent with the contact inhibition that HepG2 cells exhibit in a culture flask under standard cell culture condition (on tissue culture polystyrene), where the cells grow in a complete monolayer. In contrast, the fluorescence from the modified OBS plate seeded with equal amounts of HepG2 cells was greater than the fluorescent signal from the unmodified plate. The difference in fluorescence was significant after 6 days of culture (see FIG. 4). This increase in fluorescence is directly correlated with oxygen consumptions of the cell contained in these wells. The increase in oxygen consumption of the cells cultured on the modified surfaces indicates the HepG2 cells were not contact inhibited, were more metabolically active and were able to proliferate more than cells cultured on the unmodified plates. This experiment demonstrates covalent modification of the OBS PDMS surface with ECM proteins renders the surface more cell adhesive. The increased adhesiveness of the OBS-containing surface is thus able to mimic the surfaces of common tissue culture surfaces.

1. A method of producing a surface with enhanced cell-adhesive properties, comprising
a) treating a pre-formed surface to expose at least one intermediate reactive group on said pre-formed surface;
b) reacting said at least one intermediate reactive group to form a non-mechanical self-assembled monolayer comprising at least one reactive group; and
c) coupling at least one cell-adhesive molecule to said at least one reactive group to produce a surface with enhanced cell-adhesive properties.

2. The method of claim 1, wherein said at least one reactive group is present on said non-mechanical self-assembled monolayer immediately after said reacting said at least one intermediate reactive group.

3. The method of claim 1, wherein said at least one reactive group is not present on said non-mechanical self-assembled monolayer immediately after said reacting said at least one intermediate reactive group, and wherein a terminal group on said non-mechanical self-assembled monolayer is converted to said reactive group.

4. The method of claim 1, wherein said intermediate reactive group is a hydroxyl group.

5. The method of claim 1, wherein said pre-formed surface comprises polymer matrix.

6. The method of claim 5, wherein said polymer matrix comprises silicone.

7. The method of claim 6 wherein said silicone is polydimethyl siloxane (PDMS).

8. The method of claim 1, wherein said pre-formed surface comprises at least one oxygen-sensing compound.

9. The method of claim 8, wherein said oxygen-sensing compound is luminescent.

10. The method of claim 1, wherein said at least one reactive group comprises a reactive group selected from the group consisting of a carboxyl group, a hydroxyl group, an amide, an amino, an acyl group, an ester, an epoxy, a silane, a silanol, an aldehyde, and a sulfhydryl group.

11. The method of claim 1, wherein said reacting said at least one intermediate reactive comprises using a reactive solution.

12. The method of claim 11, wherein said reactive solution comprises a silane.

13. The method of claim 12, wherein said silane is trichlorosilane.

14. The method of claim 1, wherein said at least one cell-adhesive molecule is selected from the group consisting of a protein, a protein fragment, a polypeptide, an oligopeptide, an amino acid, a proteoglycan, a glycoprotein, a lipoprotein, a carbohydrate, a disaccharide, a polysaccharide, a nucleic acid, an oligonucleotide, a polynucleotide, a synthetic polymer, a natural polymer and combinations thereof.

15. The method of claim 14, wherein said at least one cell-adhesive molecule is selected from the group consisting of an extracellular matrix molecule, a growth factor and an antibody.

16. The method of claim 1, wherein said coupling comprises a carbodiimide.

17. The method of claim 16, wherein said carbodiimide is selected from the group consisting of ethyldimethylaminopropyl-carbodiimide, 1-cyclohexyl-3-(2-morpholinoethyl)carbodiimide and dicyclohexyl carbodiimide.

18. The method of claim 16, wherein said coupling further comprises a stabilizing agent.

19. The method of claim 18, wherein said stabilizing agent is selected from the group consisting of N-hydroxysulfosuccinimide (sulfo-NHS), hydroxysulfosuccinimide and hydroxybenzotriazolohydrate.

20. A device comprising a surface, said surface comprising
a) a polymeric matrix;
b) at least one oxygen-sensing compound; and
c) at least one cell-adhesive molecule coupled to a non-mechanical self-assembled monolayer via at least one reactive group, said non-mechanical self-assembled monolayer comprising said reactive group.
